(19) United States
(12) Patent Application Publication
(10) Pub. No.: US 2026/0054082 A1
(43) Pub. Date: Feb. 26, 2026

(54) METHODS, DEVICES, AND SYSTEMS FOR NON-INVASIVE PREVENTION AND TREATMENT OF PREMATURE EJACULATION

(71) Applicant: OHH-MED MEDICAL LTD, Tiberias (IL)

(72) Inventor: Daniel Lischinsky, Ramat Yishai (IL)

(73) Assignee: OHH-MED MEDICAL LTD, Tiberias (IL)

(21) Appl. No.: 19/304,993

(22) Filed: Aug. 20, 2025

Related U.S. Application Data
(60) Provisional application No. 63/685,279, filed on Aug. 21, 2024.

(51) Int. Cl. A61N 1/40; A61K 31/167; A61K 38/48; A61N 1/05; A61N 7/00

(52) U.S. Cl. CPC A61N 1/403; A61K 31/167; A61K 38/4893; A61N 1/0502; A61N 7/00; C12Y 304/24069; A61N 2007/0026

(57) ABSTRACT

Devices, systems, and methods are claimed for the treatment of premature ejaculation using neuromodulation, e.g., via radio frequency (RF) or ultrasound (US) energy to disrupt neural function at the dorsal penile nerves or pelvic floor. In one embodiment, a device for the non-invasive prevention or treatment of premature ejaculation is disclosed, the device being applicable externally to a penis of an individual, the device comprising: at least one radiofrequency (RF) generator configured to generate RF energy; at least one RF electrode suitable for placement on a target penile surface of the penis proximate to at least one dorsal penile nerve; and at least one control circuit connected to the RF generator, wherein the at least one control circuit is operable to cause the RF energy to selectively affect the at least one dorsal penile nerve to prolong ejaculation time.

### CROSS-REFERENCE TO RELATED APPLICATIONS

This Application is a U.S. Non-Provisional Patent Application that claims priority to U.S. Provisional Ser. No. 63/685,279 filed on Aug. 8, 2024 all of which is incorporated by reference herein in its entirety.

### RELATED APPLICATIONS

The present application is also related to co-owned PCT Patent Application 9 PCT/IL2025/050708 filed Aug. 19, 2025, titled “METHODS, DEVICES, AND SYSTEMS FORNON-INVASIVE PREVENTION AND TREATMENT OF PREMATURE EJACULATION,” Daniel Lischinsky inventor, currently assigned to Ohh-Med Medical Ltd. which is hereby incorporated by reference.

The present application is also related to co-owned U.S. Pat. No. 9,913,981 B1, titled “METHODS AND DEVICES FOR TREATING ERECTILE DYSFUNCTION,” Daniel Lischinsky inventor, currently assigned to Ohh-Med Medical Ltd. which is hereby incorporated by reference to the extent that it is not inconsistent with the instant claims, description, and drawings.

### FIELD OF THE CLAIMED INVENTIONS

The present application relates to methods, devices, and systems using electric currents induced by radio frequency generators or using ultrasound for the neuromodulation of penile nerves for the prevention and treatment of premature ejaculation; e.g., CPC class code A61N.

### SUMMARY

Devices, systems, and methods are claimed for the treatment of premature ejaculation using neuromodulation, e.g., via radio frequency (RF) or ultrasound (US) energy to permanently or temporarily disrupt neural function at the region of the dorsal penile nerves.

In one embodiment, a device for the non-invasive prevention or treatment of premature ejaculation is disclosed, the device being applicable externally to a penis of an individual, the device comprising: at least one radiofrequency (RF) generator configured to generate RF energy; at least one RF electrode suitable for placement on a target penile surface of the penis proximate to at least one dorsal penile nerve; and at least one control circuit connected to the RF generator wherein the at least one control circuit is operable to cause the RF energy to selectively affect the at least one dorsal penile nerve to prolong ejaculation time.

In another embodiment, a device comprises at least one first ultrasound (US) transducer configured to generate US energy, wherein the at least one first ultrasound (US) transducer is configured for placement on a target penile surface of the penis proximate to at least one dorsal penile nerve; at least one second ultrasound (US) transducer configured to generate US energy, wherein the at least one second ultrasound (US) transducer is configured for placement on a perineum surface; and at least one control circuit connected to the at least one first US transducer and the at least one second US transducer, wherein the at least one control circuit is operable to cause the first and second US transducers to transmit ultrasound energy through at least a portion of the at least one dorsal penile nerve and through at least a portion of pelvic floor tissue, respectively to prolong ejaculation time.

In another embodiment, a method is disclosed for the non-invasive treatment of premature ejaculation in which RF energy is applied to a target penile surface of a penis proximate to at least one dorsal penile nerve, wherein the applying RF energy to the target penile surface of the penis proximate to at least one dorsal penile nerve is operable to selectively induce an electric field in at least a portion of the at least one dorsal penile nerve to prolong ejaculation time.

### BRIEF DESCRIPTION OF THE DRAWINGS

FIG. 1 is a schematic drawing of a cross-section of a human penis, showing the left and right dorsal penile nerves, the modulation of which is the subject of this application.

FIGS. 2 and 3 are schematic drawings of a cross-section of a human penis, showing the application of radio frequency energy to a portion of the penis in the area of the dorsal penile nerves.

FIG. 4 is a schematic drawing of a lateral view of a human penis, showing the application of radio frequency energy to a portion of the penis in the area of the dorsal penile nerves, via electrodes in a ring configuration.

FIG. 5 is a schematic drawing of a lateral view of a human penis, showing the application of radio frequency energy to a portion of the penis in the area of the dorsal penile nerves, via electrodes in a dual ring configuration.

FIG. 6 is a schematic drawing of a lateral view of a human penis, showing the application of radio frequency energy to a portion of the penis in the area of the dorsal penile nerves, via electrodes in a linear or longitudinal configuration.

FIG. 7 is a schematic drawing of a portion of the perineum, showing the application of radio frequency energy to a portion of the perineum including pelvic floor tissue.

FIG. 8 is a schematic drawing of a system for the prevention or treatment of premature ejaculation, including device components in an example embodiment of the instant application.

FIGS. 9-11 are flow diagrams of a series of operations for the prevention or treatment of premature ejaculation in example embodiments of the instant application.

### BACKGROUND

Premature ejaculation or PE is when a man climaxes and ejaculates (releases semen) sooner than he would like during sex. It is a common sexual issue, occurring in about 1 in 3 men aged 18 to 59 years old in the United States. PE can be primary or acquired, and it may get worse over time.

Ejaculation has two phases: emission and expulsion.

### Phase 1: Emission

Emission is when sperm moves from the testicles to the prostate. There it mixes with seminal fluid to make semen. The vasa deferentia (vas deferens, singular), or sperm ducts, are the tubes through which the sperm moves from the testicles to the prostate to the base of the penis.

### Phase 2: Expulsion

Expulsion is when the muscles at the base of the penis contract. This forces semen out of the penis. Often, ejaculation and orgasm (climax) happen at the same time. Some men climax without ejaculating. In most cases, erections go away after this phase.

The exact cause of PE is not known, but there are many reasons why a man may have PE. There may be biological, chemical, or emotional reasons. Some possible causes include the following:

### Serotonin Imbalance

Serotonin is a natural substance in your body made by nerves. It helps to control the way the brain manages mood, emotion, sleep, and sexual desire. High amounts of serotonin in the brain increase the time to ejaculation. Low amounts can shorten the time to ejaculation, and lead to PE.

### Psychological Issues

Psychological or mental health issues can be involved with PE and may include: depression, anxiety, stress, guilt, unrealistic expectations about sex, lack of confidence, history of sexual repression (blocked or bottled-up sexual feelings), or relationship problems. See https://www.urologyhealth.org/urology-a-z/p/premature-ejaculation.

Today, there are various approaches to address PE, each with drawbacks and limitations. There are anesthetic creams to reduce nervous system sensory input, surgical methods such as dorsal neurectomy, medicinal approaches such as with SSRIs such as Dapoxetine and PDE5i (sildenafil), as well as botox injections. Psychological or behavioral therapies are also available.

### Neuromodulation Approaches

An experimental approach has been developed to disrupt penile nerve function using pulsed radiofrequency (PRF) neuromodulation. In this study, the authors positioned an RF needle electrode near each of the left and right dorsal penile nerves.

According to the research publication, a radiofrequency probe was administered invasively via cannula at the 11 o'clock position, to address the right penile nerve. Voltage was less than 0.45 volts. 50 Hz. The impedance ranged from 200 to 450 ohms. PRF procedures were performed with a setting of 2×20 ms/s with a generator output (RFG-1A Radiofrequency Generator; Cosman Medical, Inc, Burlington, Massachusetts) of 45 V for a duration of 180 seconds at 42° C. The RFK cannula was introduced into the skin of the flaccid penis at the 1 o'clock position to apply PRF to the left DPN and the same procedure was repeated on the right DPN. See A Novel Treatment Modality in Patients With Premature Ejaculation Resistant to Conventional Methods: The Neuromodulation of Dorsal Penile Nerves by Pulsed Radiofrequency, Basal et al., Journal of Andrology 2010 Mar-Apr; 31(2):126-30. https://pubmed.ncbi.nlm.nih.gov/19395368/. The needle-based, invasive nature of this approach makes it unlikely to gain widespread acceptance among patients.

Another therapeutic approach involves pelvic floor muscle rehabilitation using electro-stimulation. For example, a company called Virility Medical uses an approach in which a pad is attached to the perineum during intercourse; the pad produces electrical signals as a way of disrupting sensory signals in the pelvic floor. Clinical trial results with this technology, however, have shown only a modest improvement. See https://www.virilitymedical.com/.

Other studies have looked at similar pelvic floor muscle rehabilitation, including a) physio-kinesiotherapy of the pelvic floor to achieve a muscle contraction that allows a patient to be aware of motor activity; (b) electro-stimulation of the perineal floor to stimulate directly the pudendal nerve, resulting in stimulation of the puborectalis muscle, which causes the urethral sphincter to contract; and (c) biofeedback, in which the patient learns to control the muscle contractions of the perineal floor and the genitourinary sphincter. In one study, patients had three 60-min therapy sessions each week, during which the three techniques were applied for 20 minutes each. Again, only modest improvement was seen with these methods. See Pelvic floor muscle rehabilitation for patients with lifelong premature ejaculation: a novel therapeutic approach, Pastore et al., Therapeutic Advances in Urology. 2014;6(3): 83-88. https://journals.sagepub.com/doi/10.1177/1756287214523329.

### DETAILED DESCRIPTION

Accordingly, the instant application describes improved approaches for the treatment and prevention of premature ejaculation, applying neuromodulation technology to the penis, and to the perineum and pelvic floor, in a non-invasive fashion.

### Radiofrequency Neuromodulation Embodiments

Radiofrequency neuromodulation involves the localized application of radiofrequency energy via electrodes, often in a pulsed fashion, for a defined period of time. Effects of applying RF electrical energy to human tissue include localized temperature increases and disruption of nerve signaling.

Pulsed-radiofrequency neuromodulation (PRF) is a known pain management technique that involves placing an electrode near nerves and generating electrical current pulses in order to modulate or affect, often in an inhibitory manner, the transduction of somatosensory information through those nerves. This technique evolved from a similar radiofrequency procedure in which constant current is distributed to a nerve or neural structure. RF has been shown to have neuromodulatory effects, including interrupting nerve conduction and preventing somatosensory information from reaching the brain. In the case of continuous radiofrequency, however, the current can cause destructive nerve lesions if not controlled appropriately. PRF is generally non-destructive, safe, and effective in neuromodulation applications. See Willett, Alex, Pulsed Radiofrequency Neuromodulation of Peripheral Nerve Injury, (2011), Honors Projects, Illinois Wesleyan University, 145. https://digitalcommons.iwu.edu/psych_honproj/145/.

Various kinds of RF electrodes may be used, including single, bipolar, multipolar, or metal-based flat array electrodes (FAEs).

In some embodiments, a minimally invasive procedure for microdamaging nerves may be performed, such as Laser Interstitial Thermal Therapy (LITT), RadioFrequency Ablation (RFA), Stereotactic RadioSurgery (SRS), or Radiofrequency Microneedling (RFMN).

In one such embodiment, microneedle RF electrodes may be used to induce an electric field that affects the penile nerve(s) or pelvic floor tissue. Examples of microneedle electrodes include microneedle array electrodes (MAEs), insulated or non-insulated radiofrequency microneedling (RFMN), fractional microneedling Radiofrequency (FMR), and bipolar microneedle electrode systems. RF microneedling or other minimally invasive approaches are an effective way of delivering current to nerves, especially the superficial nerves of the penis and pelvic floor.

Each of the above RF embodiments may be seen as a stepped-down version of radiofrequency ablation, performed painlessly and without large needles.

The above techniques may be applied to the penis and pelvic floor areas in a scheduled manner, as way to permanently or semi-permanently decrease the sensitivity of the penis and thereby decrease the severity of premature ejaculation. In some embodiments, treatments are administered by a physician to empirically determine the appropriate power level and duration of the RF or US to achieve the desired effect. For example, a physician may begin RF pulse or US treatment at very low power levels, wait for feedback from the patient as to PE symptom status, and increase power level in a subsequent session until an improvement is seen in PE symptoms, e.g., longer time to ejaculation. Monitoring for stability of the neuromodulatory effect will determine whether ongoing treatment is necessary.

In some embodiments, the neuromodulation by RF or US described herein will result in slight permanent or semi-permanent damage to one or both of the dorsal penile nerves, or other nerves in the penis or pelvic floor, reducing sensitivity just enough to prolong time to ejaculation and improve PE symptoms. Accordingly, in these embodiments, the therapy is not required to be administered near the time of intercourse, but rather is a long-term treatment or even cure.

Selective application of RF or US energy to the dorsal penile nerve(s) or pelvic floor tissue will avoid or minimize effects on other nerve function or other tissue, such as blood vessels, smooth muscle, or connective tissue.

In other embodiments, treatment may be administered days or hours before intercourse in order to decrease sensitivity of penile nerves in a short-term manner. In some embodiments, a patient may administer treatment to himself in consultation with a physician as to device placement, power levels, and duration of RF energy application. Device settings will vary from patient to patient depending on the severity of the patient's PE, the anatomy of the patient, and other factors. For example, power and pulse duration settings may be adjusted depending on the dimensions of the patient's penile shaft.

### Ultrasound Neuromodulation Embodiments

Ultrasonic neuromodulation delivers low-intensity ultrasound (US) to nervous system tissue, resulting in transient modulation of neural activity. See Ultrasound Neuromodulation: A Review of Results, Mechanisms and Safety, Blackmore et al., Ultrasound Med Biol., 2019 Jul; 45(7): 1509-1536. https: //www. ncbi. nlm. nih. gov/pmc/articles/PMC6996285/ Various ultrasound technologies may be employed in order to effect neuromodulation, such as Low Intensity Pulsed Ultrasound (LIPUS).

It has been shown that ultrasound frequencies of about 400 khz may be used to stimulate the dorsal nerve of the penis or the pudendal nerve. A range of ultrasound frequency from 0.5 to 3 MHz may also be used to treat the nerves, and ultrasound frequencies may be adjusted depending on the dimensions of the patient's penile shaft. The properties of an ultrasound transducer depend upon its diameter and frequency. For example, a small diameter transducer produces a generally small diameter ultrasound beam.

In some embodiments, ultrasound may be administered using a transducer fixed inside a cuff or sleeve that can be placed comfortably on the penis during treatment. The following mechanism of action of US in neuromodulation has been proposed: when the US transducer is activated, it emits acoustic energy waves toward a nerve, such as the dorsal nerve of the penis or pudendal nerve, which cause it to be stimulated or inhibited. As the energy waves travel transdermally through the penis tissue, cavitation can occur that creates vapor cavities. These vapor cavities or small liquid-free zones (“bubbles” or “voids”) are created by rapid changes of pressure. Where the pressure is low, a void is created. When subjected to higher pressure, the voids implode and generate a shock wave. These shockwaves cause the dorsal nerve of the penis or pudendal nerve to be stimulated or inhibited. See WO2018231911A1, PCT application PCT/US2018/037222, Jeffrey Bennett and William Rissmann, inventors. https://patents.google.com/patent/WO2018231911A1/en.

In the instant application, ultrasound may be used alone as a neuromodulation agent, administered in the region of the dorsal penile nerves and the perineum; or US may be used in combination with the RF methods described above.

Combined RF and US therapy may be applied to the penile shaft alone, or the combination may be applied to the penile shaft and perineum. Alternatively, RF neuromodulation can be applied to the penile shaft, and US applied to the perineum, or vice versa.

In the following description, various aspects of the present invention are described. For purposes of explanation, specific configurations and details are set forth in order to provide a thorough understanding of the present invention. However, it will also be apparent to one skilled in the art that the present invention as claimed is not limited to the specific details presented herein. Furthermore, well known features may have been omitted or simplified so as to not obscure the present invention.

With specific reference to the drawings, it is stressed that the particulars shown are by way of example and for purposes of illustrative discussion of the present invention only, and are presented in the cause of providing what is believed to be the most useful and readily understood description of the principles and conceptual aspects of the claimed inventions. In this regard, no attempt is made to show structural details of the claimed inventions in more detail than is necessary for a fundamental understanding of the claimed inventions, the description taken with the drawings making apparent to those skilled in the art how a multitude of forms of the claimed inventions may be embodied in practice.

Before at least one embodiment of the claimed inventions are explained in detail, it is to be understood that the claimed inventions are not limited in its application to the details of construction and the arrangement of the components set forth in the following description or illustrated in the drawings. The claimed inventions are applicable to other embodiments that may be practiced or carried out in various ways as well as to combinations of the disclosed embodiments. Also, it is to be understood that the phraseology and terminology employed herein is for the purpose of description and should not be regarded as limiting.

Methods, systems, and devices for treating premature ejaculation are disclosed. Methods are aimed at reducing penile nerve sensitivity by delivering energy to specific penile tissue in the region of the penile nerves or pelvic floor. Devices may be configured to generate RF energy and to apply the generated RF energy to a penis to thereby disrupt nerve signaling or elevate a temperature of internal penile tissue above a predetermined temperature value while maintaining a penile skin surface below a predetermined temperature threshold. The predetermined temperature value may be set at a level that is effective to decrease sensory penile nerve function.

Additionally, as discussed above, ultrasound neuromodulation may also be used either alone or in combination with RF neuromodulation or other PE therapy modalities to effect optimal results. Advantageously, disclosed embodiments provide preventive, therapeutic, or curative methods and devices for long-term reduction of premature ejaculation symptoms, achieved by painless and non-invasive procedures.

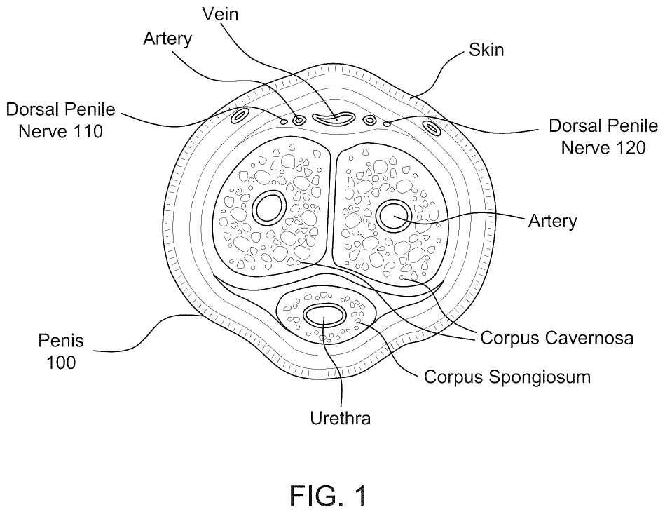

FIG. 1 is a high-level schematic illustration of a penis 100, in cross-section, illustrating, inter alia, the target regions of interest for neuromodulation at dorsal penile nerve 110 and dorsal penile nerve 120.

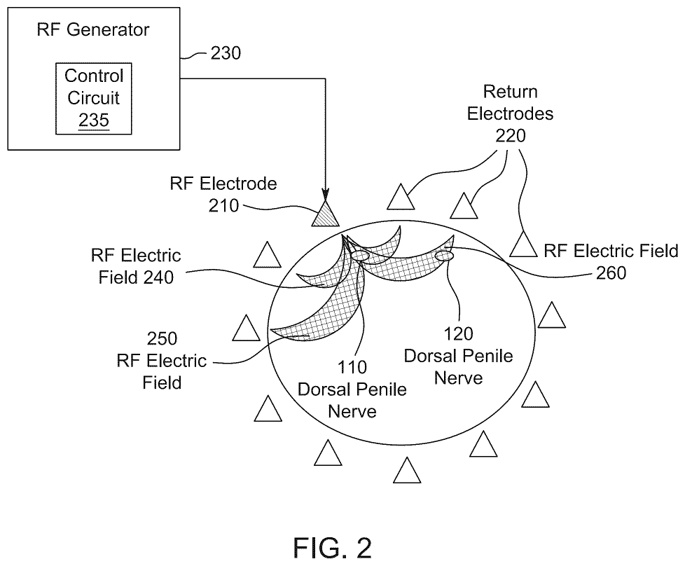

FIG. 2 is a high-level line drawing of the cross-sectional view of FIG. 1, with the components of the claimed inventions illustrated. In this embodiment, a series of electrodes are positioned around the circumference of a penis, in a ring configuration, with electrodes positioned at the hour positions of a clock face. Here, a single RF electrode 210 is positioned at 11 o'clock such that RF electrode 210 receives an RF signal from RF Generator 230 to send current through penile tissue proximate to dorsal penile nerve 110. All other electrodes function as return electrodes 220, thus creating RF electric field 240, electric field 250, electric field 260, etc., which together converge on dorsal penile nerve 110 to effectuate neuromodulation of the nerve.

Control Circuit 235 can then switch the configuration of the electrodes such that a single RF electrode 210 is positioned at 1 o'clock such that RF electrode 210 receives an RF signal from RF Generator 230 to send current through penile tissue proximate to dorsal penile nerve 120. All other electrodes function as return electrodes 220, thus creating RF electric fields that converge on dorsal penile nerve 120 to effectuate neuromodulation of the nerve.

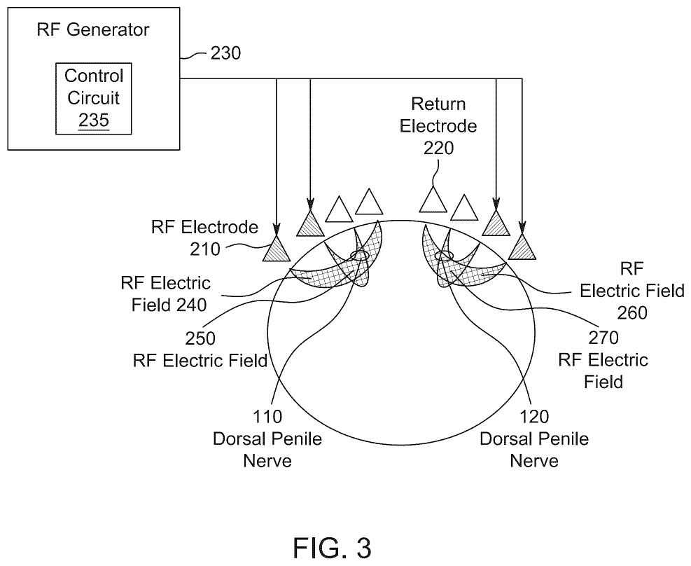

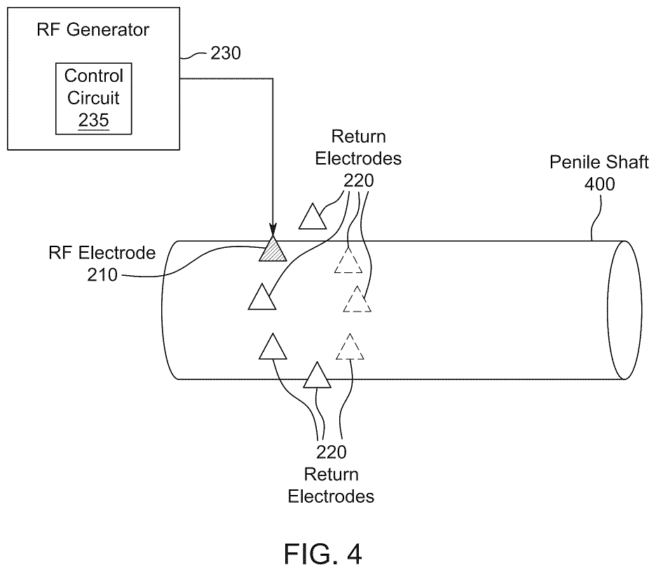

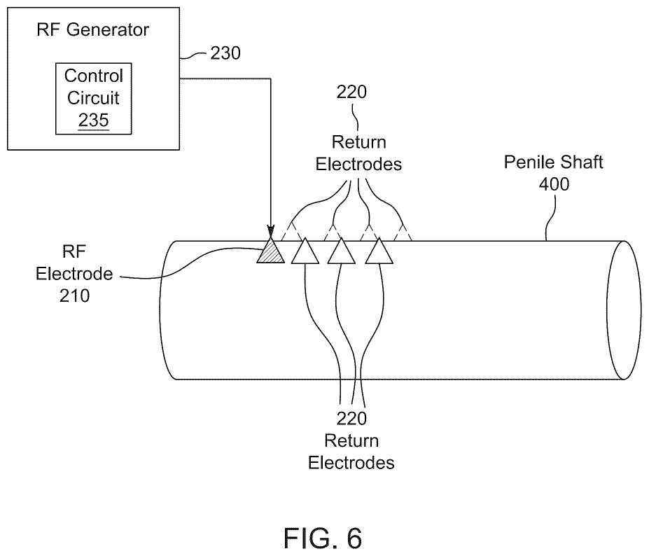

FIG. 3 is a high-level line drawing of the cross-sectional view of FIG. 1, with the components of the claimed inventions illustrated. In this embodiment, a series of electrodes are positioned in an inverted U configuration on the dorsal aspect of the penis. Here, electrode pairs are positioned such that RF electrode 210 receives an RF signal from RF Generator 230 to send current through penile tissue proximate to dorsal penile nerve 110 to return electrode 220, thus creating RF electric field 240, which intersects dorsal penile nerve 110 to effectuate neuromodulation of the nerve. This can be done with additional electrode pairs either at a similar coordinate on the circumference of the penis as shown, or at a different coordinate longitudinally along the penile shaft as shown in FIG. 6.

Power levels of RF (or ultrasound) may be varied depending on the desired effect on the individual. In some embodiments, a series of pulsed current or ultrasound applications at a certain power level may have an inhibitory effect on the penile nerves so as to disrupt neural signaling and thereby reduce sensitivity and improve PE. In other embodiments, power level and duration of RF current or ultrasound may be at least partially excitatory on the nerves, potentially inducing a refractory period in the nerves during which sensitivity is decreased and PE symptoms improved.

In some embodiments, RF generator 230 may be configured to generate RF energy at a frequency ranging between 100 KHz-40 MHz. In other embodiments, RF generator 230 may be configured to generate RF energy at power ranging between 1-100 W. In some embodiments, RF generator 230 may be configured to generate RF energy at power ranging between 15-25 W. In yet other embodiments, the power of RF energy generated by RF generator 230 may be determined based on, for example, a geometry of the RF electrodes or RF electrode pairs, such as relative distance between electrodes, or RF power may be based on tissue parameters (e.g., impedance, geometry, size, etc.) of a specific patient undergoing the treatment.

In some embodiments, RF pulse duration may range from 1 millisecond to 3 seconds. In a preferred embodiment, the pulse duration will be from 1 to 50 milliseconds. In some embodiments, a pulse or a series of pulses may be delivered over the course of up to 15 minutes. Short pulses or series of pulses should not be expected to raise tissue temperature significantly, however longer pulses may increase localized tissue temperatures to between 37 and 96 degrees Celsius. Temperature sensing as described herein will sense skin surface temperature to make sure that skin surface temperatures do not exceed 43 degrees Celsius, to avoid skin burns. The overall intent of the PE therapy is not to significantly raise the temperature of the penile tissue, but rather to shock the nerve tissue to cause microdamage that reduces penis sensitivity only as much as required to prolong ejaculation time to desired levels.

RF generator 230 may include control circuitry 235 connected to each RF electrode or each RF electrode pair. Such control circuitry 235 may be configured to control generation and delivery of generated RF energy to penis 100 via at least one RF electrode 210 or at least one return electrode 220, for example, to disrupt neural signaling or to elevate a temperature of a target internal penile tissue (e.g., the tissue at or proximate to a dorsal penile nerve, as shown in FIG. 2) above a predetermined temperature value.

In some embodiments, RF generator control circuitry 235 may be configured to control RF generator 230 to generate RF energy having predetermined energy parameters. Predetermined energy parameters may include, for example, at least one of a predetermined frequency, phase, intensity, or polarity. In some embodiments, RF generator 230 or control circuitry 235 may be configured to perform multiplexing of predetermined energy parameters to generate predetermined energy patterns. Predetermined energy patterns may include, for example, frequency patterns, phase patterns, intensity patterns, modulation patterns, or waveform pattern. In some embodiments, control circuitry 235 may be configured to operate RF electrodes or RF electrode pairs according to a predetermined operation pattern. Predetermined operation patterns may include, for example, an intensity pattern, a mode pattern, a timing pattern, or a time duration of RF electrode operation.

In various embodiments, at least one of the energy parameters, energy patterns, operation patterns or any combination thereof may be determined and controlled based on a desired depth of RF energy delivery (e.g., depending on a target inner penile nerve tissue) or based on a desired temperature value (e.g., depending on a desired treatment effect).

In some embodiments, RF electrodes need not be touching the skin, but rather may function via capacitance coupling through a film, or otherwise function through a thin layer or insulator between the electrode and the skin itself.

RF electrode and return electrode configurations may have a wide variety of arrangements to effectuate neuromodulation of penile nerve tissue or pelvic floor nerve tissue. For example, in addition to the configurations shown in the instant figures, a unipolar configuration of RF electrode without return may be used, a return path through the ground may be used, a plate return electrode may be used at any position on the body, a bipolar or tripolar RF electrode configuration may be used, any arrangement of multipolar RF electrodes may be used, any arrangement of US transducers may be used, or any arrangement of a combination of RF electrodes and US transducers may be used.

In some embodiments, as shown in FIGS. 2 and 3 (single ring configuration) and 4 (dual ring configuration), electrodes may be arranged as a ring around the circumference of penile shaft 400, with electrodes at some or all of the hour positions of a clock face relative to a cross-sectional view of the penis. Additional electrode rings may be used as desired. In these embodiments, RF generator 230 may send current to RF electrode 210 at the 11 o'clock position as a single pulse near the right dorsal penile nerve 110 (the other electrodes will function as return electrodes 220 in this configuration); and then the configuration may be changed to send current to RF electrode 210 at the 1 o'clock position as a single pulse near the right left dorsal penile nerve 120 (the other electrodes will function as return electrodes 220 in this configuration).

In some embodiments, the system will generate one pulse with the RF electrode at the 11 o'clock configuration, and then the system will automatically change the configuration to treat the other side of the penis, with the RF electrode 210 at the 1 o'clock configuration. Accordingly, at first only RF electrode 210 at the 11'oclock position is the active, while all the rest of the electrodes are return electrodes 220, and then after a pre-determined time or a manual switching by the physician or patient, the electrode at the 1'oclock position becomes the active RF electrode 210 and all the rest become return electrodes 220.

Return electrodes 220 may be positioned in a number of ways so as to induce current in the area of one or both dorsal penile nerves when paired with RF electrode 210. In some embodiments, return electrodes 220 may take the form of a plate electrode positioned on the opposite side of the penis from RF electrode 210, another position on the penis, or positioned on any other part of the body, e.g., the foot.

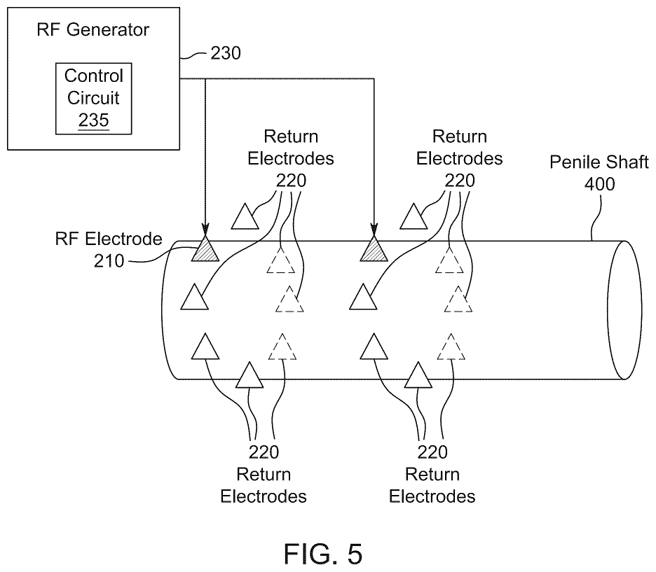

FIG. 5 illustrates a dual ring configuration having two rings of electrodes at different positions along the length of penile shaft 400. FIG. 6 illustrates a substantially linear configuration, wherein electrodes are positioned in a pairwise configuration on the dorsal aspect of penile shaft 400, on either side of the midline.

In some embodiments, a sleeve or cuff may be employed to provide support for electrodes. The sleeve or cuff may fit snugly around the penis in order to provide contact between electrodes housed therein and the skin (or any thin film between the electrode(s) and the skin). The sleeve or cuff may comprise an elastic material to facilitate fitting and removal. In one embodiment, the electrodes or transducers may be in the form of a bar that can be positioned via the sleeve or other support longitudinally along the penis, at an angle, or in another position that facilitates the appropriate delivery of energy to the dorsal penile nerves.

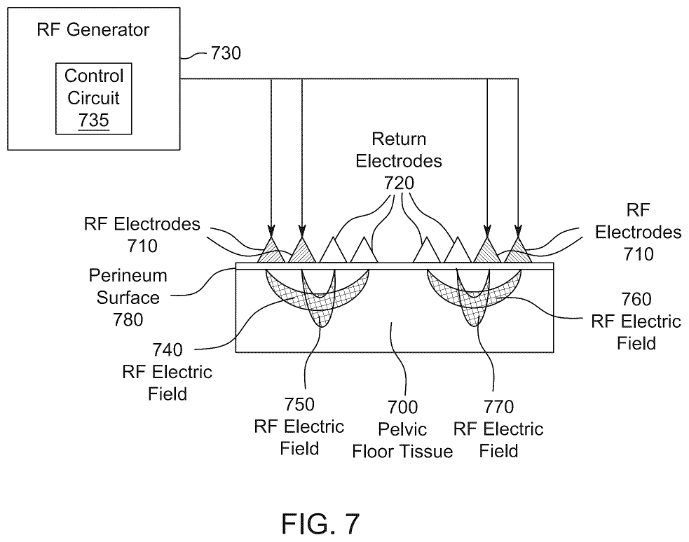

FIG. 7 illustrates a substantially two dimensional pad configuration designed for applying RF energy to the perineum. Here, electrodes are affixed within a pad made of, e.g., plastic or fabric, which, when placed in contact with the perineum will generate RF electric field 740 within pelvic floor tissue 700. Neuronal signal disruption or elevated temperature will then effectuate neuromodulation of this tissue, which may contribute additively or synergistically to penile neuromodulation, as described above.

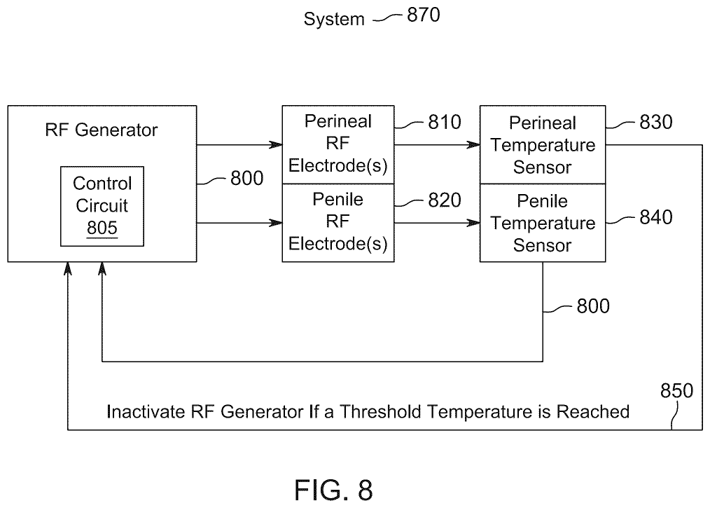

FIG. 8 shows a basic circuit diagram showing control paths and feedback control for a system 870. Here, RF generator 800 or control circuit 805 signals to Perineal RF Electrode(s) 810 and Penile RF Electrode(s) 820 to initiate neuromodulation of the respective target tissues. In some embodiments, a synchronization circuit is present to synchronize application of RF pulses to the Perineal RF Electrode(s) 810 and Penile RF Electrode(s) 820 so that they are applied at the same time, in phase. In an alternative embodiment, the synchronization circuit is operable to shift the phase of the RF pulses to Perineal RF Electrode(s) 810 and Penile RF Electrode(s) 820 so that they are applied out of phase with each other.

In some embodiments, an arrangement of Penile RF Electrode(s) 820 may be affixed in a housing, e.g., made of plastic, to facilitate positioning on, around, or near the penis. In one embodiment the arrangement of Penile RF Electrode(s) 820 affixed in a housing may include a penile temperature sensor 840 positioned on the skin proximate to the target tissue for the detection of a temperature limit. Similarly, Perineal RF Electrode(s) 810 may be affixed within a pad or other housing structure to facilitate proper positioning of electrodes and temperature sensors, if any, on the perineum. In one embodiment, the pad or other housing structure may include a perineal temperature sensor 830 positioned on the skin proximate to the target tissue for the detection of a temperature limit.

In the above examples of temperature detection, temperature may be used as a proxy signal to indicate disruption of neural signals in the target tissues. As a safety measure, a temperature threshold may be set to signal the RF generator 800 or control circuit 805 to cease operation, to prevent heat damage to target tissues.

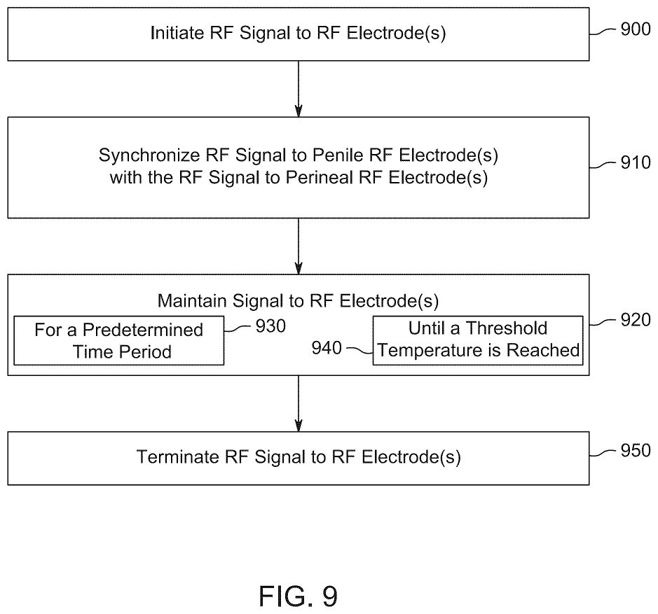

Accordingly, FIGS. 9-11 describe examples of methods or software flow diagrams showing operations that may be carried out by system 870 or an ultrasound version of system 870.

The operations shown in FIG. 9 include initiating RF signal to RF electrode(s) 900, for example by a user activating RF generator 800 or control circuit 805. Next is a synchronization of RF signal to penile RF electrode(s) with the RF signal to perineal RF electrode(s) 910, followed by maintenance of the signal to RF electrode(s) 920 for a specified duration or series of pulses. This may be continued for a predetermined time period 930 or until a threshold temperature is reached 940. When either of those conditions is met, RF generator 800 or control circuit 805 automatically terminates RF signal to RF electrode(s) 950.

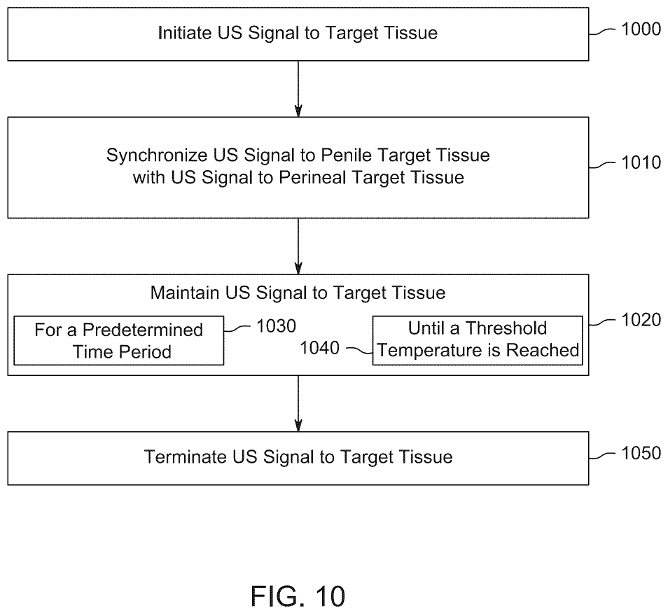

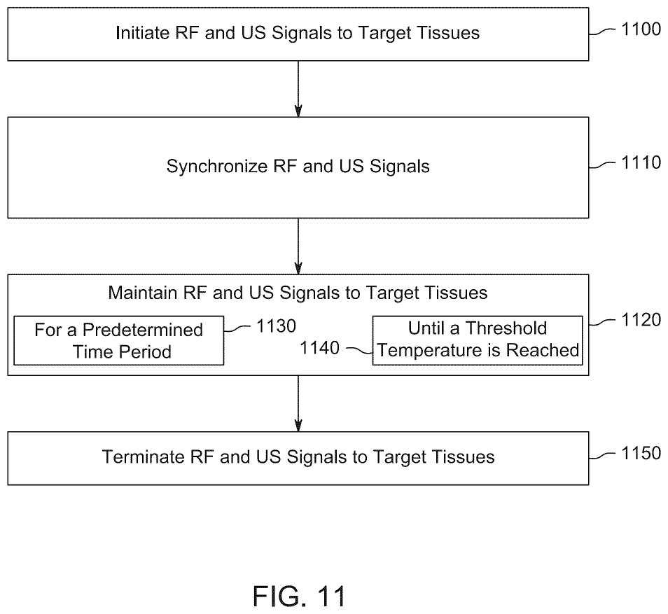

FIG. 10 illustrates a similar flow for application and control of ultrasound signal to penile and perineal target tissues, and FIG. 11 shows a similar flow where both RF and US neuromodulation are employed.

### Clauses

Clause 1: A device for the non-invasive treatment of premature ejaculation, the device being applicable externally to a penis of an individual, the device comprising:
at least one radiofrequency (RF) generator configured to generate RF energy;
at least one RF electrode suitable for placement on a target penile surface of the penis proximate to at least one dorsal penile nerve; and
at least one control circuit connected to the RF generator wherein the control circuit is operable to cause or modulate the RF energy to selectively affect the at least one dorsal penile nerve to prolong ejaculation time.

Clause 2: The device of clause 1, further comprising at least one return electrode positioned to induce an electric field in the region proximate to the at least one dorsal penile nerve when paired with the at least one RF electrode.

Clause 3: The device of clause 1, wherein the at least one control circuit is operable to cause the RF energy to selectively elevate the temperature of the region proximate to the at least one dorsal penile nerve.

Clause 4: The device of clause 1, wherein at least two RF electrodes are configured to contact the target penile surface at substantially opposing sides of the penis.

Clause 5: The device of clause 1, further comprising at least one temperature sensor configured to measure temperature at one or more target penile surfaces.

Clause 6: The device of clause 5, wherein the at least one control circuit is operable to maintain the one or more target penile surfaces below a predetermined temperature.

Clause 7: The device of clause 1, wherein a first RF electrode and a plurality of return RF electrodes are affixed to a housing such that they are positionable in a ring-shaped configuration around a circumference of the penis.

Clause 8: The device of clause 1, wherein the first RF electrode and the plurality of return electrodes are affixed to a housing such that they are positionable in a U-shaped configuration around a portion of the penis.

Clause 9: The device of clause 1, wherein the first RF electrode and the plurality of return electrodes are affixed to a housing such that they are positionable in a substantially linear configuration on a portion of the penis.

Clause 10: The device of clause 1, wherein the first RF electrode and the plurality of return electrodes are positioned in substantially any configuration on a portion of the penis.

Clause 11: The device of clause 1, further comprising a pad including one or more pad RF electrodes, wherein the pad is configured to be applied to at least a portion of a perineum of the patient, and wherein the pad RF electrodes are configured to electrically stimulate pelvic floor tissues proximate to the at least a portion of a perineum.

Clause 12: The device of clause 11, wherein the pad RF electrodes are configured to elevate the temperature of the pelvic floor tissues proximate to the at least a portion of a perineum.

Clause 13: A device for the non-invasive treatment of premature ejaculation, the device being applicable externally to a penis portion and perineum portion of a patient, the device comprising:
at least one first ultrasound (US) transducer configured to generate US energy;
   wherein the at least one first ultrasound (US) transducer is configured for placement on
   a target penile surface of the penis proximate to at least one dorsal penile nerve;
at least one second ultrasound (US) transducer configured to generate US energy;
   wherein the at least one second ultrasound (US) transducer is configured for placement on a perineum surface; and
at least one control circuit connected to the at least one first US transducer and the at least one second US transducer, wherein the at least one control circuit is operable to cause the first and second US transducers to transmit ultrasound energy through at least a portion of the at least one dorsal penile nerve and through at least a portion of pelvic floor tissue, respectively, to prolong ejaculation time.

Clause 14: The device of clause 13, wherein the at least one control circuit is operable to cause the US energy to selectively affect a) the at least a portion of the penis proximate to the at least one dorsal penile nerve; and b) the at least a portion of pelvic floor tissue.

Clause 15: A method for the non-invasive treatment of premature ejaculation, the method comprising:
applying RF energy to a target penile surface of a penis proximate to at least one dorsal penile nerve;
   wherein the applying RF energy to the target penile surface of the penis proximate to at least one dorsal penile nerve is operable to selectively induce an electric field in at least a portion of the at least one dorsal penile nerve to prolong ejaculation time.

Clause 16: The method of clause 15, wherein the induction of an electric field occurs within a range of 0.5 mm to 15 mm below the target penile surface.

Clause 17: The method of clause 15, wherein the induction of an electric field occurs for a period of time not to exceed three seconds.

Clause 18: The method of clause 15, wherein the induction of an electric field is stopped before a target temperature of the at least a portion of the at least one dorsal penile nerve is reached.

Clause 19: The method of clause 15, further comprising applying RF energy to a target perineum surface in coordination with the applying RF energy to a target penile surface.

Clause 20: The method of clause 15, further comprising the administration of at least one of: an anesthetic cream to the penis, at least one injectable anesthetic to the penis, at least one selective serotonin reuptake inhibitor, or at least one phosphodiesterase 5 inhibitor.

Clause 21: The method of clause 20, wherein the anesthetic cream comprises lidocaine cream.

Clause 22: The method of clause 20, wherein the injectable anesthetic comprises one or more of lidocaine or botulinum toxin.

Clause 23: A method for the non-invasive treatment of premature ejaculation, the method comprising:
applying ultrasound (US) energy to a target penile surface of a penis proximate to at least one dorsal penile nerve;
   wherein the applying US energy to the target penile surface of the penis proximate to at least one dorsal penile nerve is operable to selectively neuromodulate at least a portion of the at least one dorsal penile nerve to prolong ejaculation time.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

In some implementations described herein, logic and similar implementations may include software or other control structures suitable to operation. Electronic circuitry, for example, may manifest one or more paths of electrical current constructed and arranged to implement various logic functions as described herein. In some implementations, one or more medias are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively, or additionally, in some variants, an implementation may include special-purpose hardware, software, firmware components, and/or general-purpose components executing or otherwise controlling special-purpose components. Specifications or other implementations may be transmitted by one or more instances of tangible or transitory transmission media as described herein, optionally by packet transmission or otherwise by passing through distributed media at various times.

Alternatively, or additionally, implementations may include executing a special-purpose instruction sequence or otherwise operating circuitry for enabling, triggering, coordinating, requesting, or otherwise causing one or more occurrences of any functional operations described above. In some variants, operational or other logical descriptions herein may be expressed directly as source code and compiled or otherwise expressed as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar modes of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

Those having ordinary skill in the art will recognize that the state of the art has progressed to the point where there is little distinction left between hardware, software, and/or firmware implementations of aspects of systems; the use of hardware, software, and/or firmware is generally a design choice representing cost vs. efficiency tradeoffs (but not always, in that in certain contexts the choice between hardware and software can become significant). Those having ordinary skill in the art will appreciate that there are various vehicles by which processes and/or systems and/or other technologies described herein can be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be affected, none of which is inherently superior to the other in that any vehicle to be utilized is a choice dependent upon the context in which the vehicle will be deployed and the specific concerns (e.g., speed, flexibility, or predictability) of the implementer, any of which may vary.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those having ordinary skill in the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a USB drive, a solid state memory device, a hard disk drive, a Compact Disc (CD), a Digital Video Disk (DVD), a digital tape, a computer memory, etc. ; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, those skilled in the art will recognize that the various aspects described herein which can be implemented, individually and/or collectively, by a wide range of hardware, software, firmware, and/or any combination thereof can be viewed as being composed of various types of “electrical circuitry. ” Consequently, as used herein “electrical circuitry” or “control circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read-only, etc.)), and/or electrical circuitry forming a communications device (e.g., a modem, communications switch, optical-electrical equipment, etc.). Those having ordinary skill in the art will recognize that the subject matter described herein may be implemented in an analog or digital fashion or some combination thereof.

Those skilled in the art will recognize that at least a portion of the devices and/or processes described herein can be integrated into a data processing system. Those having ordinary skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

In certain cases, use of a system or method as disclosed and claimed herein may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory.

Further, implementation of at least part of a system for performing a method in one territory does not preclude use of the system in another territory.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., operations), devices, objects, and the discussion accompanying them are used as examples for the sake of conceptual clarity and that various configuration modifications are contemplated. Consequently, as used herein, the specific examples set forth and the accompanying discussion are intended to be representative of their more general classes. In general, use of any specific example is intended to be representative of its class, and the non-inclusion of specific components (e.g., operations), devices, and objects should not be taken to be limiting.

With respect to the use of substantially any plural and/or singular terms herein, those having ordinary skill in the art can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are presented merely as examples, and that in fact many other architectures may be implemented which achieve the same functionality. In a conceptual sense, any arrangement of components to achieve the same functionality is effectively “associated” such that the desired functionality is achieved. Therefore, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of “operably couplable” include but are not limited to physically mateable or physically interacting components, wirelessly interactable components, wirelessly interacting components, logically interacting components, or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components, inactive-state components, or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, the following appended claims may contain usage of the introductory phrases “at least one”and “one or more”to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such a recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having ordinary skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having ordinary skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented as sequences of operations, it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A device for the non-invasive treatment of premature ejaculation, the device being applicable externally to a penis of an individual, the device comprising:
at least one radiofrequency (RF) generator configured to generate RF energy;
at least one RF electrode suitable for placement on a target penile surface of the penis proximate to at least one dorsal penile nerve; and
at least one control circuit connected to the RF generator wherein the control circuit is operable to cause the RF energy to selectively affect the at least one dorsal penile nerve to prolong ejaculation time.

2. The device of claim 1, further comprising at least one return electrode positioned to induce an electric field in the region proximate to the at least one dorsal penile nerve when paired with the at least one RF electrode.

3. The device of claim 1, wherein the at least one control circuit is operable to cause the RF energy to selectively elevate the temperature of the region proximate to the at least one dorsal penile nerve.

4. The device of claim 1, wherein at least two RF electrodes are configured to contact the target penile surface at substantially opposing sides of the penis.

5. The device of claim 1, further comprising at least one temperature sensor configured to measure temperature at one or more target penile surfaces.

6. The device of claim 5, wherein the at least one control circuit is operable to maintain the one or more target penile surfaces below a predetermined temperature.

7. The device of claim 1, wherein a first RF electrode and a plurality of return RF electrodes are affixed to a housing such that they are positionable in a ring-shaped configuration around a circumference of the penis.

8. The device of claim 1, wherein the first RF electrode and the plurality of return electrodes are affixed to a housing such that they are positionable in a U-shaped configuration around a portion of the penis.

9. The device of claim 1, wherein the first RF electrode and the plurality of return electrodes are affixed to a housing such that they are positionable in a substantially linear configuration on a portion of the penis.

10. The device of claim 1, wherein the first RF electrode and the plurality of return electrodes are positioned in substantially any configuration on a portion of the penis.

11. The device of claim 1, wherein the at least one RF electrode comprises at least one microneedle electrode.

12. The device of claim 1, further comprising a pad including one or more pad RF electrodes, wherein the pad is configured to be applied to at least a portion of a perineum of the patient, and wherein the pad RF electrodes are configured to electrically stimulate pelvic floor tissues proximate to the at least a portion of a perineum.

13. The device of claim 12, wherein the pad RF electrodes are configured to elevate the temperature of the pelvic floor tissues proximate to the at least a portion of a perineum.

14. A device for the non-invasive treatment of premature ejaculation, the device being applicable externally to a penis portion and perineum portion of a patient, the device comprising:
at least one first ultrasound (US) transducer configured to generate US energy;
wherein the at least one first ultrasound (US) transducer is configured for placement on a target penile surface of the penis proximate to at least one dorsal penile nerve;
at least one second ultrasound (US) transducer configured to generate US energy;
wherein the at least one second ultrasound (US) transducer is configured for placement on a perineum surface; and
at least one control circuit connected to the at least one first US transducer and the at least one second US transducer, wherein the at least one control circuit is operable to cause the first and second US transducers to transmit ultrasound energy through at least a portion of the at least one dorsal penile nerve and through at least a portion of pelvic floor tissue, respectively, to prolong ejaculation time.

15. The device of claim 14, wherein the at least one control circuit is operable to cause the US energy to selectively affect a) the at least a portion of the penis proximate to the at least one dorsal penile nerve; and b) the at least a portion of pelvic floor tissue.

16. A method for the non-invasive treatment of premature ejaculation, the method comprising:
applying RF energy to a target penile surface of a penis proximate to at least one dorsal penile nerve;
wherein the applying RF energy to the target penile surface of the penis proximate to at least one dorsal penile nerve is operable to selectively induce an electric field in at least a portion of the at least one dorsal penile nerve to prolong ejaculation time.

17. The method of claim 16, wherein the induction of an electric field occurs within a range of 0.5 mm to 15 mm below the target penile surface.

18. The method of claim 16, wherein the induction of an electric field occurs for a period of time not to exceed three seconds.

19. The method of claim 16, wherein the induction of an electric field is stopped before a target temperature of the at least a portion of the at least one dorsal penile nerve is reached.

20. The method of claim 16, further comprising applying RF energy to a target perineum surface in coordination with the applying RF energy to a target penile surface.

21. The method of claim 16, further comprising the administration of at least one of:
an anesthetic cream to the penis, at least one injectable anesthetic to the penis, at least one selective serotonin reuptake inhibitor, or at least one phosphodiesterase 5 inhibitor.

22. The method of claim 21, wherein the anesthetic cream comprises lidocaine cream.

23. The method of claim 21, wherein the injectable anesthetic comprises one or more of lidocaine or botulinum toxin.
